(19) United States
(12) Patent Application Publication
(10) Pub. No.: US 2025/0195564 A1
(43) Pub. Date: Jun. 19, 2025

(54) IRON SUPPLEMENT COMPOSITIONS AND METHODS OF USE THEREOF

(71) Applicant: ThermoLife International, LLC, Signal Hill, CA (US)

(72) Inventors: Ronald KRAMER, Signal Hill, CA (US); Alexandros NIKOLAIDIS, Nea Kallikratia (GR)

(21) Appl. No.: 19/067,509

(22) Filed: Feb. 28, 2025

Related U.S. Application Data
(62) Division of application No. 17/889,244, filed on Aug. 16, 2022.
Application No. 17/889,244 is a continuation of application No. PCT/US22/40429, filed on Aug. 16, 2022.
(60) Provisional application No. 63/233,682, filed on Aug. 16, 2021.

(51) Int. Cl. A61K 33/26; A61K 9/00; A61K 9/20; A61K 31/375; A61K 36/21; A61P 43/00

(52) U.S. Cl. CPC A61K 33/26; A61K 9/0056; A61K 9/20; A61K 31/375; A61K 36/21; A61P 43/00

(57) ABSTRACT

The disclosure is directed to a method of replenishing a subject's iron, ferritin, and/or hemoglobin level. The method includes orally administering elemental metal iron to the subject; and orally administering a source of nitrate anion to the subject.

### CROSS REFERENCE TO RELATED APPLICATIONS

This application is a divisional of U.S. application Ser. No. 17/889,244, filed on Aug. 16, 2022, which is a continuation of International Application No. PCT/US22/40429, filed on Aug. 16, 2022, which claims priority to U.S. Provisional Patent Application No. 63/233,682, filed on Aug. 16, 2021, the contents of which are incorporated herein by reference in their entireties.

### BACKGROUND

Anemia is a condition in which the blood is deficient in red blood cells, in hemoglobin, or in total volume. Hemoglobin depletion can cause many debilitating symptoms, such as, extreme fatigue, weakness, pale skin, chest pain, fast heartbeat, shortness of breath, headache, dizziness or lightheadedness, cold hands and feet, inflammation or soreness of the tongue, brittle nails, unusual cravings for non-nutritive substances (such as ice, dirt, or starch), and poor appetite (especially in infants and children with iron deficiency anemia). Anemia is a prevalent issue in elder population where above normal blood pressure levels is also a prevalent issue. Other high-risk groups include pregnant women, women of reproductive age, non-Hispanic blacks, and Hispanics.

Iron-deficiency anemia (IDA) occurs when the iron deficiency is sufficient to reduce erythropoiesis and therefore the hemoglobin level falls. IDA may impact at early stages of life by causing growth retardation, lower cognitive abilities, lethargy and poor attention. This public health concern has stimulated the research for the development of alternative preventive solutions.

Iron deficiency is the most prevalent nutritional deficiency and the main cause of anemia. Infants less than 2 years and pregnant women being the most vulnerable groups. Globally, anemia affects 1.62 billion people (95% CI: 1.50-1.74 billion), which corresponds to 24.8% of the population (95% CI: 22.9-26.7%). The highest prevalence is in preschool-age children (47.4%, 95% CI: 45.7-49.1), with 43% of all pre-school children having anemia. The lowest prevalence is in men (12.7%, 95% CI: 8.6-16.9%). However, the population group with the greatest number of individuals affected is non-pregnant women (468.4 million, 95% CI: 446.2-490.6).

| | | | Population affected | |
|---|---|---|---|---|
| | Prevalence of anaemia | | Number | |
| Population group | Percent | 95% CI | (millions) | 95% CI |
| Preschool-age children | 47.4 | 45.7-49.1 | 293 | 263-303 |
| School-age children | 25.4 | 19.9-30.9 | 305 | 238-371 |
| Pregnant women | 41.8 | 39.9-43.8 | 56 | 54-59 |
| Non-pregnant women | 30.2 | 28.7-31.6 | 468 | 446-491 |
| Men | 12.7 | 8.6-15.9 | 260 | 175-345 |
| Elderly | 23.9 | 18.3-29.4 | 164 | 126-202 |
| Total population | 24.8 | 22.9-26.7 | 1620 | 1500-1740 |

Because anemia hinders the blood's ability to carry oxygen, it can severely affect pregnant women and children. In the current time of the COVID-19 pandemic, anemia is a dangerous comorbidity in pregnant women and children. Damage from anemia may be permanent and lead to the development of neoplastic processes, diabetes, cardiovascular disease.

Daily iron supplementation has been proposed as an effective remedy for older children and pregnant women, but evidence of such an effect in infants' iron deficiency is scarce. Iron supplementation is recommended as a priority strategy and has been endorsed by the World Health Organization (WHO) when the prevalence of iron deficiency is high in a specific population. Pediatric societies around the world recommend daily ferrous sulfate supplementation in infants less than 4 months of age receiving formula and from 6 months onwards in breastfed infants. Despite this recommendation and the free supply of ferrous sulfate, adherence is low, mainly due to gastrointestinal adverse effects, mothers' neglect, and low rate of prescription by healthcare team members. Additionally, there are health risks to having too much iron. Excess iron, for example from over supplementation, may cause genomic instability and thereby resulting in structural and functional alterations in proteins, lipids, and DNA. Thus, although, weekly ferrous sulfate supplementation has been proposed as an alternative preventive treatment for IDA since the nineties, the need for a safe and effective iron supplementation formulation is still great and remains unanswered.

Iron deficiency can also arise from blood donation. Blood donation results in a substantial loss of iron (200 to 250 mg) at each bleeding procedure (425 to 475 ml) and subsequent mobilization of iron from body stores. Recent reports have shown that body iron reserves generally are small and iron depletion is more frequent in blood donors than in non-donors. The lost amount of hemoglobin recovered after blood donation is usually recovered after a mean of 36±11 days. Iron supplementation, in the form of usual iron salts, such as iron sulfate, can shorten the time of hemoglobin recovery to 32 days, but that benefit is minor. Thus, there is a need for an improved iron supplement formulation that can replete the body's stored hemoglobin rapidly, especially after blood donation.

### SUMMARY

Described herein is an iron supplement composition comprising elemental metal iron and a source of nitrate anion (NO₃⁻). The term “elemental metal iron” refers to elemental iron or iron in its metallic, uncharged, elemental form. The term “elemental metal iron” does not refer to any charged form of iron, such as iron oxides, iron salts, iron hydroxides, or iron hydrides. In some aspects, the source of nitrate anion is a salt of nitric acid or salt of nitrous acid, for example, potassium nitrate, sodium nitrate, magnesium nitrate, potassium nitrite, sodium nitrite, or magnesium nitrite. In other aspects, the source of nitrate anion is a botanical source, for example an Amaranthus extract. In certain embodiments, the amount of elemental metal iron is 10 mg and the amount of the source of nitrate anion provides 350 mg nitrate anion. In some aspects, the composition further comprises an acid, for example ascorbic acid. In some embodiments, the amount of acid is the composition is 100 mg.

In certain embodiments, the composition is in the dosage form of a capsule, a cachet, a pill, a tablet, a powder, a granule, a pellet, a bead, a particle, a troche, or a pastille. For example, the elemental metal and the source of nitrate anion are in separate dosage forms. In other implementations, the elemental metal and the source of nitrate anion are packaged in a capsule, a cachet, a pill, a tablet, a powder, a granule, a pellet, a bead, a particle, a troche, or a pastille. In certain embodiments, the acid is packaged separately from the source of nitrate anion and the elemental metal. In some aspects, the source of nitrate anion, the elemental metal, and the acid are in solid forms.

Also described are methods of iron supplementation, for example, methods of replenishing iron and/or ferritin in a subject or methods of replenishing hemoglobin storage in a subject. In some aspects, the subject is diagnosed with anemia, low blood iron level, low ferritin level, and/or low hemoglobin levels. In some implementations, the subject exhibits at least one symptom selected from the group consisting of: extreme fatigue, weakness, pale skin, chest pain, fast heartbeat, shortness of breath, headache, dizziness or lightheadedness, cold hands and feet, inflammation or soreness of the tongue, brittle nails, unusual cravings for non-nutritive substance, and poor appetite. Also described are methods of replenishing blood iron level, ferritin level, and/or hemoglobin levels in a subject, for example, after a subject a suffer has suffered a blood loss incident, which includes a blood donation. These methods comprise administering to a subject the iron supplement composition described herein.

Thus, the method comprises orally administering an effective amount of elemental metal iron to the subject and orally administering an effective amount of a source of nitrate anion to the subject. In certain implementations, the effective amount of elemental metal iron is 1-100 mg. The effective amount of nitrate anion is 30-4000 mg. In some aspects, the method also comprises orally administering an effective amount of an acid in the subject. The effective amount of the acid is 10-5000 mg.

In some implementations, the effective amount of the elemental metal iron is 0.3-45 μmol/kg body weight and the effective amount of the nitrate anion is 0.008-1.6 mmol/kg body weight. Where the acid is ascorbic acid, the effective amount of the acid is 0.001-0.7 mmol/kg body weight. In a particular embodiment, the effective amount of the elemental metal iron is 0.0032 mmol/kg body weight, the effective amount of the nitrate anion is 0.1000 mmol/kg body weight, and the effective amount of the ascorbic acid is 0.0101 mmol/kg body weight.

### DETAILED DESCRIPTION

Detailed aspects and applications of the disclosure are described below in the following detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant art, that implementations of the technology disclosed herein may be practiced without these specific details. It should be noted that there are many different and alternative configurations, devices, and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

As used herein, the term “about” refers to a deviation no more than 5% of the given value, for example a deviation of 3%, 2%, 1%, 0.5%, or 0.1% of the given value.

As used herein, the term “acceptable” is a phrase used in its broadest sense and may describe ingredients of a composition that meet Food and Drug Administration (FDA) standards, United States Pharmacopeia (USP) standards, US Department of Agriculture (USDA) standards for food-grade materials, commonly accepted standards of the nutritional supplement industry, industry standards, botanical standards, or standards established by any individual. These standards may delineate acceptable ranges of aspects of ingredients of a composition such as edibility, toxicity, pharmacological effect, or any other aspect of a chemical, composition, or preparation used in implementations of a composition.

As used herein, the term “composition” refers to both a mixture of ingredients or constituents as well as a combination of capsules that contains different ingredients or constituents. Accordingly, in certain embodiments, a composition encompasses separate capsules that are packaged together and are meant to be taken together.

As used herein, the term “elemental metal iron” refers to the neutral-charged state of iron, in other words, iron in its elemental form and not in a salt form or charged form (exemplary salt forms and charged forms include the oxide, hydroxide, carbonate, chloride, lactate, citrate, aspartate, glycinate, and gluconate of the metal). Thus, the term “elemental metal iron” does not refer to iron oxides, iron salts, iron hydroxides, or iron hydrides.

As used, herein, the term “subject” refers to a mammal, e.g., a human, mouse, rat, guinea pig, dog, cat, horse, cow, pig, or non-human primate, such as a monkey, chimpanzee, baboon, or rhesus.

The present disclosure relates to the discovery that a composition comprising elemental metal iron can improve a body's hemoglobin stores, contrary to the iron supplement products available today. The composition comprises elemental metal iron, a source of nitrate anion, and an acid. In some embodiments, the composition comprises 1-100 mg elemental metal iron, 30-4000 mg nitrate anion, and 10-5000 mg acid. In some implementations, the composition further comprises a source of folate and is can be tolerated by a pregnant subject, which can save the lives and health of millions of mothers and fetuses. In certain embodiments, the source of nitrate anion is a nitrate salt, for example potassium nitrate. In other embodiments, the source of nitrate anion is a botanical source, for example an extract of a green leafy vegetable or a plant containing high levels of nitrate such as beetroot or Amaranthus.

In some embodiments, the composition comprises about 10 mg elemental metal iron and an amount of the source of nitrate anion (NO₃⁻) that provides about 350 mg nitrate anion. The elemental metal iron may be in any form, for example, a powder or granules. In certain embodiments, the nitrate anion is provided by a botanical source, for example, an Amaranthus extract. In particular embodiments, the composition comprises about 10 mg elemental metal iron and an amount of extract from Amaranthus dubius and Amaranthus tricolor that provides about 350 mg nitrate anion (NO₃⁻). In some aspects, the composition further comprises about 100 mg of an acid, for example ascorbic acid.

In other embodiments, the composition comprises about 16.7 mg elemental metal iron and about 255 mg nitrate anion (NO₃⁻). In some aspects, such compositions further comprise a source of folate (for example, 5-methyltetrahydrofolate, 5-MTHF) and an acid. In particular embodiments, the composition further comprises about 5 mg 5-MTHF (or the equivalent of about 3 mg folate, according to the FDA-accepted conversion factor of 1.7) and about 833 mg ascorbic acid. In certain implementations, the nitrate anion (NO₃⁻) is provided in the form of a nitrate salt, for example potassium nitrate. Accordingly, in certain embodiments, the composition comprises about 16.66 mg elemental metal iron, about 416 mg potassium nitrate, about 833 mg ascorbic acid, and about 5 mg 5-MTHF.

The disclosed composition may be in the form of a capsule, tablet, pill, liquid, liquid suspension, vapor, powder, granulate, pulverulence, or a combination thereof. In a preferred embodiment, the disclosed composition is in a solid form. In certain implementations the composition is provided via a plurality of a capsule, a cachet, a pill, a tablet, a powder, a granule, a pellet, a bead, a particle, a troche, or a pastille. In some embodiments, the elemental metal iron and the source of nitrate anion (NO₃⁻), and in some aspects the acid, are combined into one capsule or one tablet. In other embodiments, the acid is in a separate tablet or capsule than the elemental metal iron and the source of the nitrate ion. The enhanced activity of co-administration the source of nitrate anion (NO₃⁻) and the elemental metal iron are not reduced if the subject ingests the source of nitrate anion (NO₃⁻) and the elemental metal iron separately, for example via co-administration of separate capsules of the source of nitrate anion (NO₃⁻) and the elemental metal iron.

Accordingly, in some aspects, the composition described herein comprises a capsule comprising a source of nitrate anion (NO₃⁻) and a capsule comprising an elemental metal iron. In another embodiment, the composition is in a capsule comprising about 2.5 mg elemental metal iron and an amount of the source of nitrate anion (NO₃⁻) that provides about 87.5 mg nitrate anion. In embodiments of the composition further comprising an acid, the composition further comprises a capsule comprising the acid. Accordingly, in some embodiments, the composition is a capsule, a cachet, a pill, a tablet, a pellet, a troche, or a pastille that comprises about 2.5 mg elemental metal iron, an amount of the source of nitrate anion that provides about 85.7 mg nitrate anion, and about 25 mg acid.

In some embodiments, the elemental metal iron in the composition is covered or microencapsulated with a suitable material that is poorly soluble in water but soluble in the acidic environment of the stomach, for example, magnesium oxide, cellulose polymers, alginates (such as calcium alginate), or aluminum hydroxide.

In some aspects, the composition further comprises a suitable pharmaceutically acceptable coating (to prevent moisture from getting into the tablets) and/or an additive. Non-limiting examples of the pharmaceutically acceptable coating include waxes, polymers, solid fatty acids, etc. Non-limiting examples of the additive include a carrier, excipient, binder, colorant, flavoring agent, preservative, buffer, diluent, and combinations thereof. In some aspects, the additive is a pharmaceutically acceptable additive or an acceptable food additive.

As shown in the examples, ingestion of the composition optimizes the body's circulation, for example, by repleting the body's storage of iron and hemoglobin. The restoration of blood iron and hemoglobin levels can occur within 24 hours of the administration of the composition. Thus, also described herein are methods of iron supplementation, for example, by replenishing iron and/or hemoglobin storage in a subject. In some aspects, the subject is diagnosed with anemia, low blood iron level, low ferritin level, and/or low hemoglobin levels. In some implementations, the subject exhibits at least one symptom selected from the group consisting of: extreme fatigue, weakness, pale skin, chest pain, fast heartbeat, shortness of breath, headache, dizziness or lightheadedness, cold hands and feet, inflammation or soreness of the tongue, brittle nails, pica (unusual cravings for non-nutritive substance), and poor appetite. Also described are methods of replenishing blood iron level, ferritin level, and/or hemoglobin levels in a subject, for example, after a subject has suffered a blood loss incident. In some aspects, the blood loss incident is a blood donation.

The oral and parental iron supplement formulations commonly used today involve a plethora of gastrointestinal side effects, such as diarrhea, stomach pain, heartburn, nausea, vomiting, black stools, and constipation. However, iron supplementation using the elemental metal iron compositions disclosed herein cause the subject to experience less gastrointestinal side effects. As shown in the examples, the subjects did not report any, let alone gastrointestinal, side effects associated with iron supplementation when they ingested the disclosed composition comprising elemental metal iron. In particular, the subject who previously did experience such side effects with an iron supplement comprising iron salts reported that she did not experience any of those side effects with the disclosed composition comprising elemental metal iron. This is particularly surprising since ingestion of nitrate salts has been well-documented to cause gastrointestinal irritation (See Berge et al., “Pharmaceutical Salts, Review Article,” J Pharm Sci., 1977, 66(1): 1-19). In fact, The Handbook of Pharmaceutical Salts explicitly recommends that nitrate salts should not be considered for pharmaceuticals used internally (see page 298 of Stahl, P. H., Wermuth, C. G. 2002. Handbook of pharmaceutical salts: Properties, selection, and use. Weinheim: VHCA). Instead, the subject reported that oral co-administration of elemental metal iron and a nitrate salt did not lead to any gastric distress.

In certain implementations, the method of replenishing blood iron level, ferritin level, and/or hemoglobin levels in a subject comprises administering to the subject an effective amount of elemental metal iron, an effective amount of nitrate anion, and, in some aspects, an effective amount of an acid. In certain embodiments, the source of nitrate anion is a nitrate salt, for example potassium nitrate. In other embodiments, the source of nitrate anion is a botanical source, for example an extract of a green leafy vegetable or a plant containing high levels of nitrate such as beetroot or Amaranthus. In some embodiments, the acid is ascorbic acid. The effective amount of elemental metal iron is about 1 mg to about 100 mg. The effective amount of nitrate anion is about 30 mg to about 4000 mg. The effective amount of the acid is about 10 mg to about 5000 mg. In some aspects, the molar ratio of the effective amount of elemental metal iron, nitrate anion, and acid is 1:5.2:0.5 to 1:28.75:3.

In some implementations, the effective amount of the elemental metal iron is 0.3-45 μmol/kg body weight, for example, 0.4-45 μmol/kg body weight, 0.3-32 μmol/kg body weight, 0.4-32 μmol/kg body weight, 0.3-30 μmol/kg body weight, 0.4-30 μmol/kg body weight, 1.5-4.8 μmol/kg body weight, 1.5-3.2 μmol/kg body weight, or 3.2-4.8 μmol/kg body weight. In certain embodiments, the effective amount of the elemental metal iron is 0.002-0.0025 mmol/kg body weight, 0.003-0.0024 mmol/kg body weight, 0.004-0.022 mmol/kg body weight, 0.005-0.020 mmol/kg body weight, or 0.007-0.030 mmol/kg body weight.

In some implementations, the effective amount of the nitrate anion is 0.008-1.6 mmol/kg body weight, for example, 0.008-1.5 mmol/kg body weight, 0.008-1.2 mmol/kg body weight, 0.01-1.6 mmol/kg body weight, 0.01-1.5 mmol/kg body weight, or 0.01-1.2 mmol/kg body weight. In certain embodiments, the effective amount of the nitrate anion is 0.05-1.0 mmol/kg body weight, 0.07-1.0 mmol/kg body weight, 0.08-1.0 mmol/kg body weight, 0.9-1.0 mmol/kg body weight, or 0.1-1.0 mmol/kg body weight, 0.05-0.12 mmol/kg body weight, 0.07-0.115 mmol/kg body weight, 0.08-0.125 mmol/kg body weight, 0.9-0.12 mmol/kg body weight, or 0.1-0.11 mmol/kg body weight.

Where the acid is ascorbic acid, the effective amount of the acid is 1.0-700 μmol/kg body weight, for example, 1.0-680 μmol/kg body weight, 1.0-510 μmol/kg body weight, 1.0-500 μmol/kg body weight, 1.3-700 μmol/kg body weight, 1.3-680 μmol/kg body weight, 1.3-510 μmol/kg body weight, 1.3-500 μmol/kg body weight. In certain embodiments, the effective amount of the ascorbic acid is 0.005-0.015 mmol/kg body weight, 0.007-0.013 mmol/kg body weight, 0.009-0.012 mmol/kg body weight, 0.01-0.012 mmol/kg body weight, 0.005-0.15 mmol/kg body weight, 0.007-0.13 mmol/kg body weight, 0.009-0.12 mmol/kg body weight, or 0.01-0.12 mmol/kg body weight.

In particular implementations, the effective amount of the elemental metal iron is 0.0032 mmol/kg body weight, the effective amount of the nitrate anion is 0.1000 mmol/kg body weight, and the effective amount of the ascorbic acid is 0.0101 mmol/kg body weight. In other implementations, the effective amount of the elemental metal iron is 0.0073 mmol/kg body weight, the effective amount of the nitrate anion is 0.1000 mmol/kg body weight, and the effective amount of the ascorbic acid is 0.115 mmol/kg body weight.

In some aspects, the effective amount of the elemental metal iron administered is 2.78-3.59 μmol/kg body weight; the effective amount of the nitrate anion administered is 86.8-113 μmol/kg body weight; and the effective amount of the ascorbic acid administered is 8.77-114 μmol/kg body weight.

In some aspects, the amount of element iron administered is the Recommended Dietary Allowance (RDA). For all ages of men and for postmenopausal women, the amount of elemental metal iron administered is 8 mg/day, which corresponds to about 143 μmol/day. Given that the average weight for American men is about 90 kg, the amount of elemental administered according to the described method may be 1.59-1.61 μmol/kg body weight when the subject is an adult male. Given that the average weight for American postmenopausal women is about 80 kg, the amount of elemental metal iron administered according to the described method may be 1.79-1.81 μmol/kg body weight when the subject is postmenopausal. The RDA for premenopausal women is 18 mg/day, which corresponds to about 322 μmol/day. Given that the average weight for American premenopausal women is about 77 kg, the amount of elemental metal iron administered according to the described method may be 4.16-4.18 μmol/kg body weight when the subject is premenopausal.

The disclosed method of replenishing blood iron level, ferritin level, and/or hemoglobin levels results in reduced gastrointestinal side effects (for example, diarrhea, stomach pain, heartburn, nausea, vomiting, black stools, and constipation) compared to conventional forms of iron supplementation, such as through oral administration of an iron salt. In some aspects, the subject experiences no side effects from the disclosed method of replenishing blood iron level, ferritin level, and/or hemoglobin levels.

### EXAMPLES

The disclosure is further illustrated by the following examples that should not be construed as limiting. The contents of all references, patents, and published patent applications cited throughout this application, as well as the Figures, are incorporated herein by reference in their entirety for all purposes.

### Example 1

One dose of the exemplary iron composition comprises 10 mg elemental metal iron, 350 mg nitrate anion, and 100 mg ascorbic acid. The dose is administered by ingestion of four gelatin capsules. Accordingly, each capsule contains 2.5 mg elemental metal iron, 87.5 mg nitrate anion, and 25 mg ascorbic acid. For subjects below average in weight, the dose for administration could be reduced to two or three gelatin capsules.

### Example 2

The hemoglobin level of a healthy Caucasian subject (40 years old male, 240 lbs) was measured using a 3-in-1 Easytouch Glucose Cholesterol Hemoglobin Monitor. The detected hemoglobin level was 16.0 g/dL, which falls within the normal range. The normal range of hemoglobin level is 3.5 to 17.5 g/dl for men and 12.0 to 15.5 g/dl for women.

The next afternoon, the subject donated one pint blood to the Red Cross. His hemoglobin levels decreased by 4.375% to 15.3 g/dL. The subject ingested one dose of the iron composition described in Example 1 with one glass of water (volume about 330 ml) after having donated blood. The next morning, the subject's hemoglobin level rose to 17.3 g/dL.

During the course of one evening, the iron supplement composition replenished and increased the subject's original hemoglobin levels when compared to the level before blood donation. This is the first observation of an iron supplement that could increase hemoglobin level (by 13%) within one day after blood donation, from 15.3 g/dL to 17.3 g/dL.

### Example 3

A pregnant female subject (40 years old, Jewish ancestry) exhibited symptoms of iron/hemoglobin deficiency. She exhibited symptoms of fatigue, weakness, pale or yellowish skin, irregular heartbeats, shortness of breath, dizziness or lightheadedness, chest pain, cold hands and feet and headache. She had previously given birth to 8 healthy children and never before experienced low iron levels. The subject tried various medicine and supplements comprising various iron salts but had to stop due to severe gastrointestinal disturbances (for example, stomach pain, heartburn, constipation, diarrhea, black stools, and nausea). Her blood was tested using a 3-in-1 Easytouch Glucose Cholesterol Hemoglobin Monitor, and the measured hemoglobin level was diagnosed as low (<7).

The patient was given a formulation comprising 833 mg of ascorbic acid, 416 mg potassium nitrate, and 16.66 mg of elemental metal iron. The subject was advised to ingest the capsules of the formulation once in the morning on an empty stomach with 500 mg cold water.

The average mesh size of the elemental metal iron was 325. The size was selected based on these iron particles dissolving under the effect of the stomach acid and the ascorbic acid in the formula slowly, which would minimize any of the GI side effects. As the subject is pregnant, a source of folate was added to the formulation as folate is highly needed for both healthy fetal development and proper hemoglobin differentiation. Given her lackluster of response to the previous iron supplementation regime, 5-MTHF (5 mg) was added the formulation instead of folate in case she lacks the ability to metabolize folic acid to 5-MTFH which is necessary for hemoglobin formation.

After five days of ingesting the formulation as advised, the patient reported noticeable improvement in feelings of well-being and health. She also reported an end to the symptoms of anemia and that she did not experience any of the GI side effects associated with her previous iron supplementation regime. Her blood hemoglobin levels were tested again after five days of supplementation, and it increased to 12.4, which is well within the normal range for a pregnant woman.

We claim:

1. A method of replenishing a subject's iron, ferritin, and/or hemoglobin level, the method comprising:
orally administering elemental metal iron to the subject; and
orally administering a source of nitrate anion to the subject.

2. The method of claim 1, wherein the source of nitrate anion is a nitrate salt.

3. The method of claim 2, wherein the source of nitrate anion is potassium nitrate.

4. The method of claim 1, wherein the source of nitrate anion is a botanical source.

5. The method of claim 4, wherein the botanical source is an Amaranthus extract or beetroot extract.

6. The method of claim 1, wherein the elemental metal iron has a mesh size of 325.

7. The method of claim 1, further comprising orally administering an acid to the subject.

8. The method of claim 7, wherein the subject is orally administered a composition comprising the elemental metal iron; source of nitrate anion; and the acid.

9. The method of claim 1 wherein the subject is diagnosed with anemia, low blood iron level, low ferritin level, and/or low hemoglobin levels.

10. The method of claim 9, wherein the subject exhibits at least one symptom selected from the group consisting of: extreme fatigue, weakness, pale skin, chest pain, fast heartbeat, shortness of breath, headache, dizziness, lightheadedness, cold extremities, tongue inflammation, tongue soreness, brittle nails, pica, and poor appetite.

11. The method of claim 9, wherein the subject's hemoglobin level is reduced from donating blood.

12. The method of claim 1, wherein the subject has donated blood.

13. The method of claim 12, wherein the subject's hemoglobin level is lower than that before donating blood.

14. The method of claim 1, where the subject exhibits reduced gastrointestinal side effect compared to oral administration of iron salts or elemental iron alone.

15. The method of claim 14, wherein the gastrointestinal side effect is at least one side effect selected from the group consisting of: diarrhea, stomach pain, heartburn, nausea, vomiting, black stools, and constipation.

16. The method of claim 1, where the subject exhibits no side effects from oral administration of the elemental metal iron and the source of nitrate anion.

17. The method of claim 1, wherein the subject is administered 10 mg elemental metal iron and 87.5 mg nitrate anion, the method further comprising orally administering 100 mg ascorbic acid to the subject.

18. The method of claim 1, wherein the subject is administered 0.004-0.22 mmol elemental metal iron/kg body weight and 0.08-1.2 mmol nitrate anion/kg body weight.

19. The method of claim 18, wherein method further comprises orally administering to the subject 0.009-0.12 mmol ascorbic acid/kg body weight.

20. The method of claim 19, wherein the subject is administered 0.0032 mmol elemental metal iron/kg body weight, 0.1000 mmol nitrate anion/kg body weight, and 0.0101 mmol ascorbic acid/kg body weight.

21. A method of increasing oral bioavailability of elemental metal iron, the method comprising:
orally administering elemental metal iron to the subject; and
orally administering a source of nitrate anion to the subject, wherein the elemental metal iron and the source of nitrate anion are co-administered.

22. The method of claim 21, wherein the source of nitrate anion is a nitrate salt.

23. The method of claim 22, wherein the source of nitrate anion is potassium nitrate.

24. The method of claim 21, wherein the source of nitrate anion is a botanical source.

25. The method of claim 24, wherein the botanical source is an Amaranthus extract or beetroot extract.

26. The method of claim 21, wherein the elemental metal iron has a mesh size of 325.

27. The method of claim 21, further comprising orally administering an acid to the subject.

28. The method of claim 27, wherein the acid is co-administered with the elemental metal iron and the source of nitrate anion.

29. The method of claim 28, wherein the subject is orally administered a composition comprising the elemental metal iron; the source of nitrate anion; and the acid.
